(19) United States
(12) Patent Application Publication
(10) Pub. No.: US 2015/0344327 A1
(43) Pub. Date: Dec. 3, 2015

(54) Hybrid water treatment agent of biogenic manganese oxide nanoparticles and activated carbon, preparation method thereof, and water treatment system and on-site underground water treatment system using the same

(71) Applicant: University-Industry Cooperation Group of Kyung Hee University, Seoul (KR)

(72) Inventors: Seok-Oh Ko, Seongnam (KR); Seoktae Kang, Yongin (KR); Do-Gun Kim, Suwon (KR)

(21) Appl. No.: 14/726,933

(22) Filed: Jun. 1, 2015

(30) Foreign Application Priority Data
May 30, 2014 (KR) 10-2014-0065630

(51) Int. Cl. C02F 1/28; B01J 20/06; B01J 20/28; C12P 3/00; B01J 35/10; B01J 37/36; B01J 37/04; B01J 20/20; B01J 23/34

(52) U.S. Cl. CPC C02F 1/288; C02F 1/281; B01J 20/20; B01J 20/06; B01J 20/28061; B01J 20/28059; B01J 23/34; B01J 35/1014; B01J 35/1019; B01J 37/36; B01J 37/04; C12P 3/00; C02F 2101/20

(57) ABSTRACT

The present invention relates to a relates to a water treatment agent, a preparation method thereof, and a water treatment system and an on-site underground water treatment system using the same, and more particularly, to a water treatment agent including an activated carbon support, and manganese oxide nanoparticles immobilized onto the activated carbon support, having a particle size less than or equal to 1,000 nm, and formed by respiration and metabolism of manganese oxidizing bacteria, a preparation method thereof, and a water treatment system and an on-site underground water treatment system using the same.

### CROSS REFERENCE TO RELATED APPLICATIONS

The application claims priority to Korean Application No. 10-2014-0065630 filed on May 30, 2014, which is incorporated herein by reference.

### TECHNICAL FIELD

The present disclosure relates to a hybrid water treatment agent of biogenic manganese oxide (BMO) nanoparticles and activated carbon, a preparation method thereof, and a water treatment system and an on-site underground water treatment system using the same, and more particularly, to a hybrid water treatment agent including an activated carbon support having BMO nanoparticles immobilized thereon, with excellent removal performance of toxic trace elements and organic pollutants in water, a preparation method thereof, and a water treatment system and an on-site underground water treatment system using the same.

### BACKGROUND ART

With the development of industrial society, various types of pollutants have been increasing in water from streams, lakes, and underground. Thus, as an amount of water resources of good quality reduces, drinking water production becomes more difficult, and available water resources reduces, the need to re-use treated wastewater dramatically increased.

Particularly, in recent years, toxic trace elements such as heavy metals, pharmaceuticals and personal care products (PPCPs), and endocrine disrupting chemicals (EDCs) increase in water from streams, lakes, and underground, and effluent in treated wastewater, which threatens the production drinking water of good quality and water re-use. Because toxicity of toxic trace elements is high even at a low concentration, toxic trace elements must be removed in a water purification process and a water recycling process. Typical examples of heavy metals include lead, cadmium, and chrome, typical examples of pharmaceuticals and personal care products include ibuprofen, acetaminophen, oxytetracycline, and caffeine, and typical examples of endocrine disrupting chemicals include 17α-ethinylestradiol (EE2), 17β-estradiol (E2), estrone (E1), and bisphenol A (BPA).

As opposed to other pollutants, toxic trace elements are not removed in general water purification and sewage and wastewater treatment processes. To remove toxic trace elements, coagulation, adsorption, biological treatment, ion exchange, membranes, and advanced oxidation processes (AOPs) are being attempted. However, they have problems such as low efficiency, high costs and secondary pollution, which limit the commercialization. Particularly, activated carbon is primarily used in a water purification process and a sewage and wastewater treatment process, and activated carbon has good adsorption performance of organic pollutants, but when organic pollutants more than a maximum adsorption capacity are adsorbed (saturated), saturated activated carbon cannot adsorb and remove an amount of pollutants any more. The saturated activated carbon needs to be replaced with new activated carbon. It is possible to reactivate saturated activated carbon, but a large amount of energy and costs is required for reactivation, secondary pollutants are created, and a partial loss occurs during reactivation. Also, activated carbon is known as having low adsorption performance of toxic trace elements.

Recently, to remove toxic trace elements, metal oxide nanoparticles are gaining attention. Metal oxide nanoparticles generally refer to particles having an individual particle size of 100 nm or less, and iron oxide, iron oxide and iron hydroxide, titanium oxide, and manganese oxide particles are mainly being studied. In the removal of pollutants in the water, attempts are being made to use metal oxide nanoparticles in a variety of application, for example, catalysts, adsorbents, and ion exchange materials, and in particular, more recently, attention is being paid to metal oxide nanoparticles as heterogeneous catalysts or adsorbents for organic pollutants oxidation/reduction.

Advantages of metal oxide nanoparticles are as follows. Firstly, a specific surface area is large, reactive sites and adsorption sites are rich on the surface, and organics oxidative removal performance and heavy metals adsorptive removal performance is very high. For example, research has reported that iron oxide nanoparticles have a phenol removal rate about 35 times higher than and an ethylene glycol removal rate 2 times to 4 times higher than the Fenton oxidation process traditionally used to remove non-biodegradable organics (Zelmanov and Semiat, 2008). Also, it was reported that micro-sized zinc oxide is incapable of adsorb and remove arsenic (As), but nano-sized zinc oxide has good arsenic adsorption and removal performance (Tiwari et al., 2008). Secondly, because pollutant removal efficiency per unit weight is high due to a large specific surface area, when removing the same amount of pollutants, an amount of injection is lower than particles having a larger particle size, so it may be used with economic efficiency.

However, metal oxide nanoparticles are difficult to commercialize due to the following drawbacks. Firstly, it takes a large amount of energy and toxic chemicals such as acid or alkali, an oxidant or a reducing agent, and a dispersant to prepare metal oxide nanoparticles, so there is a high likelihood that environment pollution and a safety-related problem will occur in the preparation process. Secondly, because nanoparticles have a small size less than or equal to 100 nm, they are difficult to separate by precipitation and filtration. That is, when nanoparticles are fed into a reactor for water treatment, they are released into water bodies together with treated water after treatment of pollutants and contaminate water from streams, lakes, and underground.

### SUMMARY OF THE DISCLOSURE

The present disclosure is designed to providing a water treatment agent including manganese oxide nanoparticles formed by respiration and metabolism of manganese oxidizing bacteria, the water treatment agent being prepared in an environmentally friendly manner and having better adsorption and oxidative removal performance of heavy metals and organic pollutants than chemically prepared metal oxide particles, in which the manganese oxide nanoparticles are immobilized and supported on an activated carbon support to prevent them from being leaked out of a reactor, the water treatment agent being applicable to a commercialization process, a preparation method thereof, and a water treatment system and an on-site underground water treatment system using the same.

To achieve the object, according to one aspect of the present disclosure, there is provided a water treatment agent including an activated carbon support, and manganese oxide nanoparticles immobilized onto the activated carbon support, having a particle size less than or equal to 1,000 nm, and formed by respiration and metabolism of manganese oxidizing bacteria.

In this instance, the manganese oxide nanoparticles may have a size of from 1 nm to 100 nm.

Also, the manganese oxide nanoparticles may be formed singularly, or by agglomerating at least two particles.

Also, the manganese oxide nanoparticles may have a specific surface area of from 80 m²/g to 300 m²/g.

Also, the manganese oxidizing bacteria may be any one or at least two selected from the group consisting of Bacillus sp. strain SG1, Leptothrix discophora strain SS-1, Pseudomonas putida strain MnB1, Pseudomonas putida strain GB-1, and Bacillus sp. WH4.

According to another aspect of the present disclosure, there is provided a method of preparing a water treatment agent, including (S1) mixing an activated carbon support having manganese ions adsorbed thereon, manganese oxidizing bacteria, and a culture medium to prepare a mixed solution, and (S2) exposing the mixed solution to room temperature and aerobic condition to oxidize the manganese ions through respiration and metabolism of the manganese oxidizing bacteria, to form an activated carbon support having manganese oxide nanoparticles of a particle size less than or equal to 1,000 nm immobilized thereon.

Here, the mixed solution in the step (S1) may have a hydrogen ion concentration of from pH 5.5 to pH 8.5.

Also, the room temperature may be from 22° C. to 28° C., and the aerobic state may have an oxygen concentration of from 1 mg/l to 10 mg/l in the mixed solution.

According to still another aspect of the present disclosure, there is provided a water treatment system including a reaction tank, wherein the reaction tank is filled with the water treatment agent of the present disclosure.

According to further another aspect of the present disclosure, there is provided an on-site underground water treatment system including a permeable reactive barrier, wherein the permeable reactive barrier is filled with the water treatment agent of the present disclosure.

According to the present disclosure, with the activated carbon support having biogenic manganese oxide (BMO) nanoparticles immobilized thereon, adsorptive removal and oxidative removal may be concurrently achieved, thereby efficiently removing toxic trace elements in water.

Also, because BMO nanoparticles are supported on an activated carbon support, a loss of the BMO nanoparticles is prevented, leading to application to a commercialization process.

Also, because BMO nanoparticles are prepared in an environmentally friendly manner, occurrence of pollutants may be minimized during a preparation process.

Also, pollutant removal performance is better than chemically synthesized manganese oxide nanoparticles.

Further, because activated carbon is used as a support, removal performance of organic pollutants is especially superior to that of the case using a traditional support.

### BRIEF DESCRIPTION OF THE DRAWINGS

The accompanying drawings illustrate a preferred embodiment of the present disclosure and together with the foregoing disclosure, serve to provide further understanding of the technical spirit of the present disclosure, and thus, the present disclosure is not construed as being limited to the drawings.

FIG. 1 shows a scanning electron microscope (SEM) image and an energy dispersive spectroscopy (EDS) graph of an activated carbon support according to one embodiment of the present disclosure.

FIG. 2 shows a SEM image and an EDS graph of an activated carbon support having manganese oxide nanoparticles immobilized thereon, the manganese oxide nanoparticles formed by respiration and metabolism of manganese oxidizing bacteria as prepared according to one embodiment of the present disclosure.

FIG. 3 is a graph showing treatment test results of an endocrine disrupting chemical 17α-ethinylestradiol (EE2) using activated carbon (AC), a water treatment agent (BMO/AC) according to one embodiment of the present disclosure, zeolite, and a water treatment agent (BMO/Zeolite) having BMO nanoparticles immobilized onto a zeolite support.

FIG. 4 is a graph showing treatment test results of heavy metal lead (Pb) using activated carbon (AC) and a water treatment agent (BMO/AC) according to one embodiment of the present disclosure.

FIG. 5 is a flowchart showing a method of preparing a water treatment agent according to one embodiment of the present disclosure.

FIG. 6 is a schematic diagram showing a water treatment system including a reaction tank filled with a water treatment agent according to one embodiment of the present disclosure.

FIG. 7 is a schematic diagram showing an on-site underground water treatment system including a permeable reactive barrier filled with a water treatment agent according to one embodiment of the present disclosure.

### DETAILED DESCRIPTION OF THE INVENTION

Hereinafter, the present disclosure is described in detail. It should be understood that the terms used in the specification and the appended claims should not be construed as limited to general and dictionary meanings, but interpreted based on the meanings and concepts corresponding to technical aspects of the present disclosure on the basis of the principle that the inventor is allowed to define terms appropriately for the best explanation.

The description proposed herein is just a preferable example for the purpose of illustrations only, not intended to limit the scope of the disclosure, so it should be understood that other equivalents and modifications could be made thereto without departing from the spirit and scope of the disclosure.

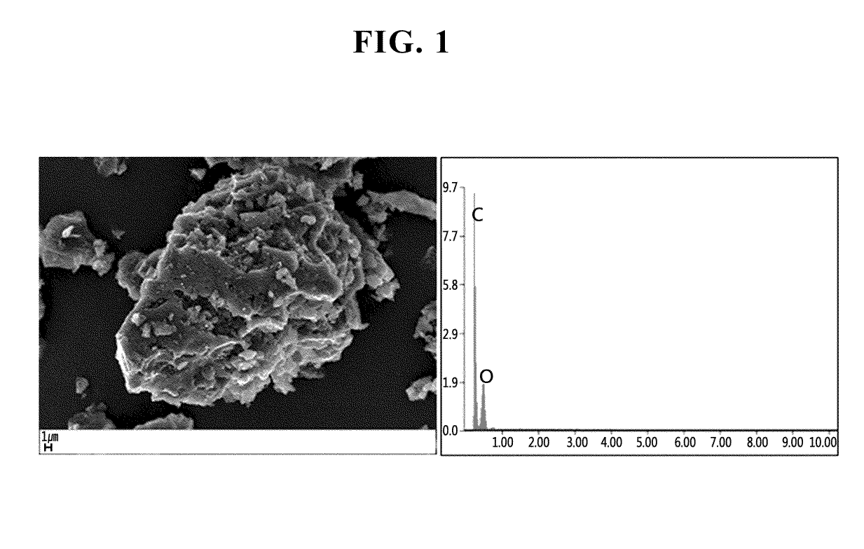

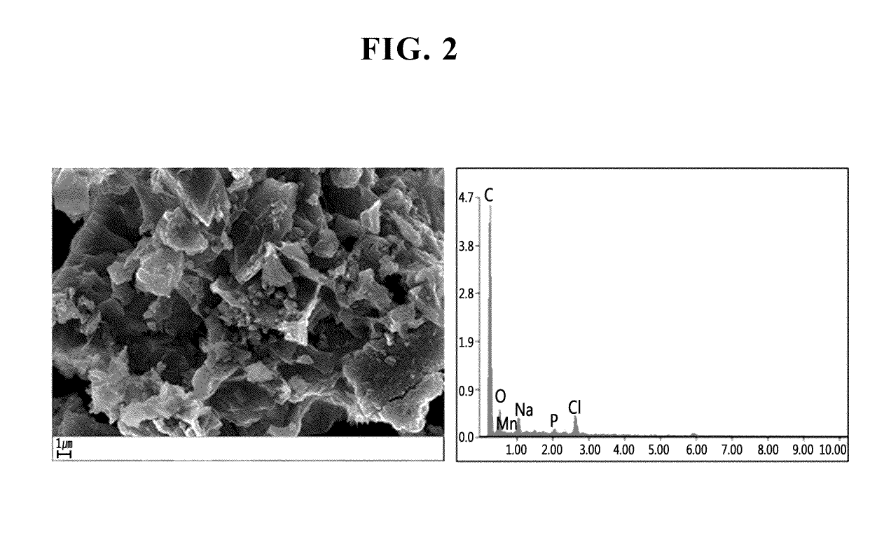

FIG. 1 shows a scanning electron microscope (SEM) image and an energy dispersive spectroscopy (EDS) graph of an activated carbon support according to one embodiment of the present disclosure, and FIG. 2 shows a SEM image and an EDS graph of an activated carbon support having manganese oxide nanoparticles immobilized thereon, the manganese oxide nanoparticles formed by respiration and metabolism of manganese oxidizing bacteria as prepared according to one embodiment of the present disclosure.

Hereinafter, referring to FIGS. 1 and 2, a water treatment agent of the present disclosure includes an activated carbon support; and manganese oxide nanoparticles immobilized onto the activated carbon support, having a particle size less than or equal to 1,000 nm, and formed by respiration and metabolism of manganese oxidizing bacteria.

The manganese oxide nanoparticles have a large specific surface area, rich reactive sites and adsorption sites on the surface, and very high organics oxidative removal performance and heavy metals adsorptive removal performance. Especially, biogenic manganese oxide (BMO) nanoparticles formed by respiration and metabolism of manganese oxidizing bacteria have much superior heavy metals adsorptive removal performance.

The following table 1 shows a comparison of amounts of adsorption of heavy metals between BMO nanoparticles and chemically synthesized manganese oxide nanoparticles.

TABLE 1

| | | | Maximum adsorption amount (mg/mg) | | |
|---|---|---|---|---|---|
| | Adsorption | | | Chemically synthesized | |
| Heavy | test | BMO nanoparticles | BMO | manganese oxide | |
| metals | conditions | producing microorganisms | nanoparticles | nanoparticles (Birnessite) | References |
| Pb | pH 6.0, 25° C. | L. discophora | 2.072 | 0.4761 | Nelson et al., 1999 |
| | pH 6.0, 25° C. | P. putida MnB1 | 1.017 | — | Villalobos et al., 2005 |
| | pH 6.0, 25° C. | P. putida MnB1 | 3.599 | 0.554 | Inventor(s) |
| Cd | pH 6.0, 25° C. | Bacillus sp. WH4 | 0.8913 | 0.080 (Todorokite) | Meng et al., 2009 |
| | pH 6.0, 25° C. | P. putida MnB1 | 1.638 | 0.202 | Inventor(s) |
| Zn | pH 6.0, 25° C. | Acremonium sp. KR21-2 | 0.1729 | 0.0468 | Tani et al., 2004 |
| | pH 6.0, 25° C. | P. putida MnB1 | 0.5714 | — | Toner et al., 2006 |
| | pH 6.0, 25° C. | P. putida MnB1 | 0.866 | 0.117 | Inventor(s) |
| Co | pH 6.0, 25° C. | Acremonium sp. KR21-2 | 0.203 | 0.0063 | Tani et al., 2004 |
| | pH 6.0, 25° C. | Paraconiothyrium sp. WL-2 | 0.108 | — | Sasaki et al., 2008 |

Referring to the above table 1, in relation to adsorption performance of heavy metals such as lead, cadmium, zinc and cobalt, BMO nanoparticles are up to about 30 times better than chemically synthesized manganese oxide nanoparticles.

Further, BMO nanoparticles is a good catalyst for organic pollutants oxidative removal capable of removing 14 types of endocrine disrupting chemicals and pharmaceuticals and personal care products in a treated sewage effluent to a maximum of 52 to 95% (Forrez et al., 2011).

However, BMO nanoparticles are currently in an initial stage of research, and laboratory-scale studies have been conducted, and because they have a problem with a loss in a real reactor due to their small size, they could not be used in a practical water treatment process.

In this context, the present disclosure solved the problem with a loss in a reactor by immobilizing and supporting the BMO nanoparticles on an activated carbon support.

Here, the active carbon (AC) is a material with strong adsorption ability, the majority of which is composed of carbonaceous materials. Particularly, due to using a support of the BMO nanoparticles as activated carbon, the present disclosure has especially better removal performance of organic pollutants than that of the case using traditional other supports.

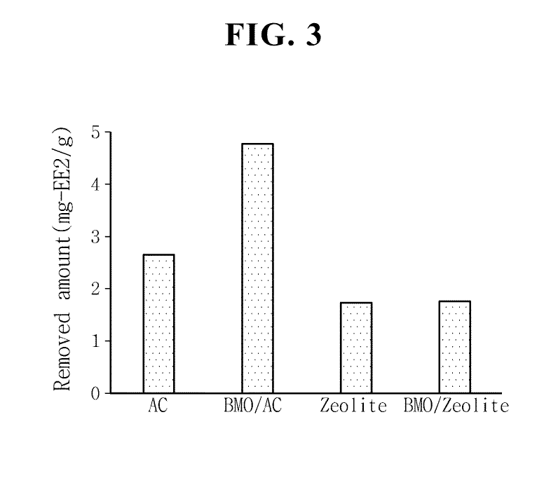

FIG. 3 is a graph showing treatment test results of an endocrine disrupting chemical 17α-ethinylestradiol (EE2) using activated carbon (AC), a water treatment agent (BMO/AC) according to one embodiment of the present disclosure, zeolite, and a water treatment agent (BMO/Zeolite) having BMO nanoparticles immobilized onto a zeolite support.

Referring to FIG. 3, BMO/AC including 0.585 mg/g of supported BMO nanoparticles had improved removal performance of 17α-ethinylestradiol (EE2) by 80.1% when compared to traditional activated carbon (AC). In contrast, when an equal amount of BMO nanoparticles were supported on zeolite, removal performance of EE2 increased by only about 1%.

The results are numerically indicated in the following table 2.

TABLE 2

| | Water treatment agent | | | |
|---|---|---|---|---|
| | AC | BMO/AC | Zeolite | BMO/Zeolite |
| EE2 removal | 2.635 | 4.746 | 1.723 | 1.740 |
| amount | | | | |
| (mg-EE2/g) | | | | |

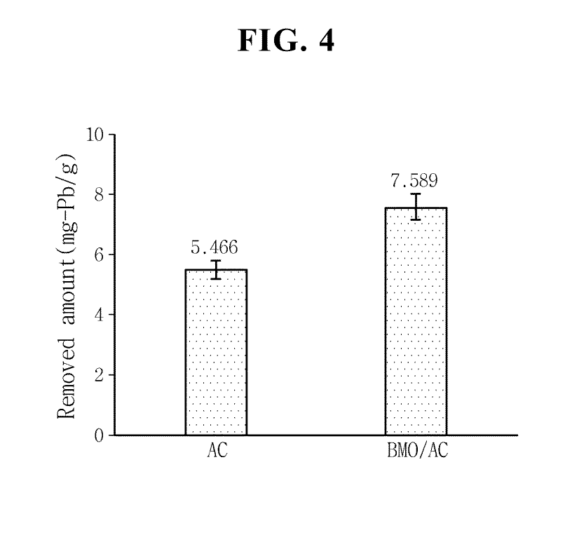

Also, FIG. 4 is a graph showing treatment test results of heavy metal lead (Pb) using activated carbon (AC) and a water treatment agent (BMO/AC) according to one embodiment of the present disclosure.

Referring to FIG. 4, the water treatment agent (BMO/AC) according to the present disclosure had higher lead removal performance by 38.9% than traditional activated carbon (AC).

On the other hand, the BMO nanoparticles may have a size of from 1 nm to 100 nm. When the numerical range is satisfied, adsorption performance of heavy metals or organic pollutants is further improved.

Also, the manganese oxide nanoparticles may be formed singularly, but may be generally formed by agglomerating at least two particles.

Also, the manganese oxide nanoparticles have a specific surface area of from 80 m²/g to 300 m²/g. In contrast, chemically synthesized manganese oxide nanoparticles have a specific surface area of from about 20 m²/g to 50 m²/g, and because the BMO nanoparticles of the present disclosure have a much larger specific surface area than chemically synthesized one as described above, their adsorption performance is far superior.

Meanwhile, the manganese oxidizing bacteria represents bacteria which oxidizes manganese ions to produce manganese oxide during respiration and metabolism, and may include, but is not limited to, any one or at least two selected from the group consisting of Bacillus sp. strain SG1, Leptothrix discophora strain SS-1, Pseudomonas putida strain MnB1, Pseudomonas putida strain GB-1 and Bacillus sp. WH4.

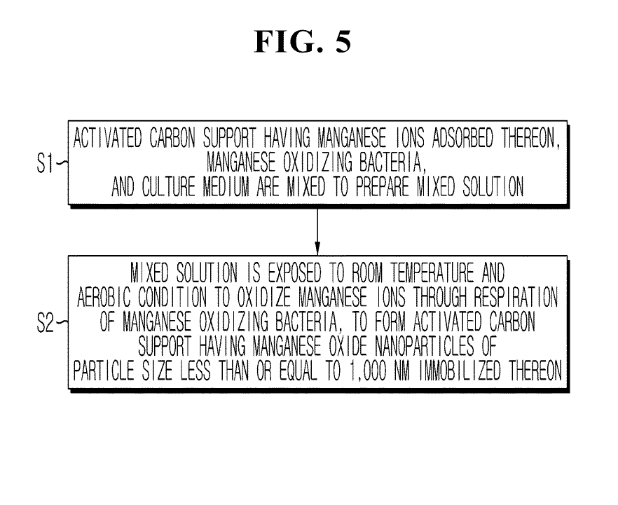

FIG. 5 is a flowchart showing a method of preparing a water treatment agent according to one embodiment of the present disclosure. Hereinafter, the method of preparing a water treatment agent according to the present disclosure is described with reference to FIG. 5.

First, an activated carbon support having manganese ions adsorbed thereon, manganese oxidizing bacteria, and a culture medium are mixed to prepare a mixed solution (S1).

In this instance, the mixed solution may have a hydrogen ion concentration of from pH 5.5 to pH 8.5, and if necessary, pH may be adjusted by adding hydrochloric acid, sulfuric acid, nitric acid, and sodium hydroxide.

Here, in connection with the activated carbon support having manganese ions adsorbed thereon, the manganese ions (Mn²⁺) are a precursor material of BMO nanoparticles to be produced later, and to adsorb manganese ions onto the activated carbon support, salts including manganese ions may be mixed with the activated carbon support to prepare a suspension, and the manganese ions may be adsorbed onto the activated carbon support.

In this instance, to increase an amount of adsorption of manganese ions onto the activated carbon support, the activated carbon support may be pre-treated using acids, alkalis, and salts, and any type of activated carbon may be used as a support irrespective of the type of activated carbon.

A method of pre-treating the activated carbon support is as follows.

First, an activated carbon support to be pre-treated is impregnated with an aqueous solution such as hydrochloric acid (HCl), nitric acid (HNO₃), sulfuric acid (H₂SO₄), sodium chloride (NaCl), sodium hydroxide (NaOH) for a predetermined period of time. Subsequently, the impregnated activated carbon support is separated from the aqueous solution by precipitation or filtration. Subsequently, the separated activated carbon support is dried, yielding a pre-treated activated carbon support.

Also, the salts including manganese ions are not limited to a particular type if they are not toxic, and specific examples include MnCl₂ and MnSO₄.

Meanwhile, as impurities other than the activated carbon support having manganese ions adsorbed thereon may be contained in the suspension, only the activated carbon support having manganese ions adsorbed thereon may be separated from the suspension and then dried.

Also, the manganese oxidizing bacteria may be the same as those described above, and the culture medium is not limited to a particular type if it is suitable for growth and proliferation of the manganese oxidizing bacteria, and various types of culture media are available.

Subsequently, the mixed solution is exposed to room temperature and aerobic condition to oxidize the manganese ions through respiration and metabolism of the manganese oxidizing bacteria, to form an activated carbon support having manganese oxide nanoparticles of a particle size less than or equal to 1,000 nm immobilized thereon (S2).

Here, the manganese oxidizing bacteria grows and proliferates by ingesting substances contained in the culture medium and respiring oxygen present in the mixed solution, and produces enzymes needed to oxidize manganese ions, and when the manganese ions adsorbed onto the activated carbon support are oxidized, manganese oxide nanoparticles are formed.

In this instance, the room temperature condition is optimum temperature condition necessary for cultivation of the manganese oxidizing bacteria, and may be from 22° C. to 28° C., more preferably from 24° C. to 27° C., but is not limited thereto.

Also, the aerobic state refers to a condition in which the bacteria may normally live or grow in the place where oxygen exists, and may be a state in which an oxygen concentration in the mixed solution is from 1 mg/l to 10 mg/l.

On the other hand, in addition to the activated carbon support having manganese oxide nanoparticles immobilized thereon, the product after the step (S2) may include the manganese oxidizing bacteria, remaining nutrient materials in the culture medium, and reaction by-products by the manganese oxidizing bacteria, and when they are removed and washing and drying is performed, only the activated carbon support having manganese oxide nanoparticles immobilized thereon may be separated.

The activated carbon support having manganese oxide nanoparticles immobilized as described above may be used in various fields including general water treatment, water recycling, and treatment of soils and underground water, and related markets are wide.

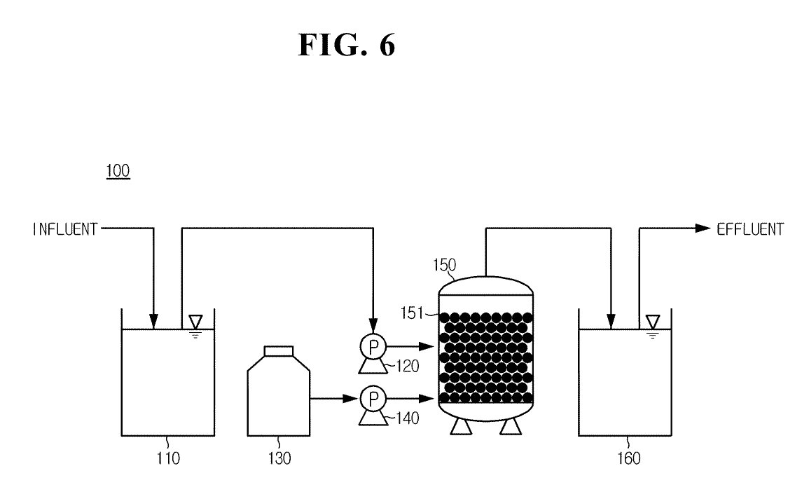

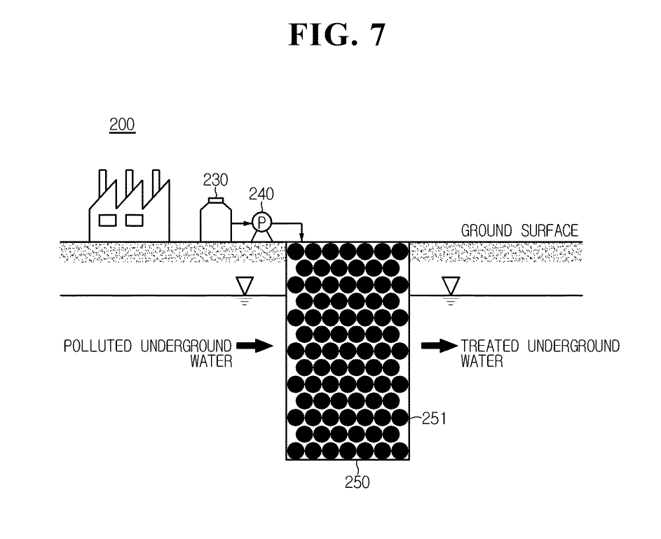

FIGS. 6 and 7 are schematic diagrams respectively showing a water treatment system including a reaction tank and an on-site underground water treatment system including a permeable reactive barrier.

Hereinafter, referring to FIGS. 6 and 7, for the water treatment system 100 including the reaction tank 150 according to the present disclosure, the reaction tank 150 is filled with a water treatment agent 151 according to the present disclosure. Also, for the on-site underground water treatment system 200 including the permeable reactive barrier 250 according to the present disclosure, the permeable reactive barrier 250 is filled with a water treatment agent 251 according to the present disclosure.

As described above, the water treatment agent according to the present disclosure may be applied to various water treatment including surface water and groundwater treatment and sewage recycling. In this instance, surface water and groundwater is used as a water source for a water purification process, and for water recycling, removal of toxic trace elements is required to produce safe recycled water.

Particularly, recently, water resources are in shortage due to water resource pollution, climate change, and population growth and water demand exceeds water supply, so the world's population suffering water shortages is about 0.7 billion people in 2008 and will be about 3 billion people by 2025 (UN, 2007). Therefore, for the benefit of supply of water resources of good quality in sufficient amount, there is an urgent need for appropriate treatment technology of toxic trace elements in all water purification and sewage/wastewater treatment, and the water treatment agent according to the present disclosure may be a solution to the above problem.

While the embodiments of the present disclosure disclosed hereinabove present merely particular examples to help the understanding, such embodiments are not intended to limit the scope of the present disclosure. It is obvious to those skilled in the art that in addition to the disclosed embodiments, modifications may be made based on the technical features of the present disclosure.

Description of Reference Numerals

| | |
|---|---|
| 100: water treatment system | 110: water collection tank |
| 120: pump | 130, 230: chemicals tank |
| 140, 240: chemicals pump | 150: reaction tank |
| 151: water treatment agent | 160: treated water tank |
| 200: on-site underground water | |
| treatment system | |
| 250: permeable reactive barrier | |
| 251: water treatment agent | |

What is claimed is:

1. A water treatment agent comprising:
an activated carbon support; and
manganese oxide nanoparticles immobilized onto the activated carbon support, having a particle size less than or equal to 1,000 nm, and formed by respiration and metabolism of manganese oxidizing bacteria.

2. The water treatment agent according to claim 1, wherein the manganese oxide nanoparticles have a size of from 1 nm to 100 nm.

3. The water treatment agent according to claim 1, wherein the manganese oxide nanoparticles are formed singularly, or by agglomerating at least two particles.

4. The water treatment agent according to claim 1, wherein the manganese oxide nanoparticles have a specific surface area of from 80 m²/g to 300 m²/g.

5. The water treatment agent according to claim 1, wherein the manganese oxidizing bacteria is any one or at least two selected from the group consisting of Bacillus sp. strain SG1, Leptothrix discophora strain SS-1, Pseudomonas putida strain MnB1, Pseudomonas putida strain GB-1, and Bacillus sp. WH4.

6. A method of preparing a water treatment agent, comprising:
(S1) mixing an activated carbon support having manganese ions adsorbed thereon, manganese oxidizing bacteria, and a culture medium to prepare a mixed solution; and
(S2) exposing the mixed solution to room temperature and aerobic condition to oxidize the manganese ions through respiration and metabolism of the manganese oxidizing bacteria, to form an activated carbon support having manganese oxide nanoparticles of a particle size less than or equal to 1,000 nm immobilized thereon.

7. The method of preparing a water treatment agent according to claim 6, wherein the manganese oxidizing bacteria is any one or at least two selected from the group consisting of Bacillus sp. strain SG1, Leptothrix discophora strain SS-1, Pseudomonas putida strain MnB1, Pseudomonas putida strain GB-1, and Bacillus sp. WH4.

8. The method of preparing a water treatment agent according to claim 6, wherein the mixed solution in the step (S1) has a hydrogen ion concentration of from pH 5.5 to pH 8.5.

9. The method of preparing a water treatment agent according to claim 6, wherein the room temperature is from 22° C. to 28° C.

10. The method of preparing a water treatment agent according to claim 6, wherein the aerobic state has an oxygen concentration of from 1 mg/l to 10 mg/l in the mixed solution.

11. A water treatment system comprising a reaction tank, wherein the reaction tank is filled with a water treatment agent according to any one of claims 1 through 5.

12. An on-site underground water treatment system comprising a permeable reactive barrier, wherein the permeable reactive barrier is filled with a water treatment agent according to any one of claims 1 through 5.
